(19) United States
(12) Patent Application Publication
(10) Pub. No.: US 2005/0239085 A1
(43) Pub. Date: Oct. 27, 2005

(54) Methods for nucleic acid sequence determination

(76) Inventors: Philip Richard Buzby, Brockton, MA (US); Rebecca Adele Ickes, Boston, MA (US); James Joseph DiMeo, Needham, MA (US)

Correspondence Address: PROSKAUER ROSE LLP, ONE INTERNATIONAL PLACE 14TH FL, BOSTON, MA, 02110, US

(21) Appl. No.: 10/831,214

(22) Filed: Apr. 23, 2004

(51) Int. Cl. C12Q 1/68; C12P 19/34

(52) U.S. Cl. 435/6; 435/91.2

(57) ABSTRACT

Methods of the invention comprise methods for nucleic acid sequence determination. Generally, the invention relates to sequencing a target nucleic acid by exposing the target nucleic acid to a primer and a polymerase. Such methods may involve determining the sequence of a target nucleic acid by using a thermophilic polymerase, such as a variant of said 9° N DNA polymerase.

### TECHNICAL FIELD OF THE INVENTION

The present invention relates to methods for nucleic acid sequence determination. More specifically, the present invention relates to sequencing a target nucleic acid by exposing the target nucleic acid to a primer and a polymerase, such as a thermophilic polymerase.

### BACKGROUND OF THE INVENTION

One of the most significant milestones in scientific history was the sequencing of the human genome. While the completion of the first human genome sequence is an important scientific milestone, many challenges remain in the areas of genetics and medicine. It is apparent that a true understanding of genetic function lies in the small variations in sequence that occur both within and between individuals. For example, relatively small genomic changes, such as single nucleotide polymorphisms, have been found to lead to profound changes in phenotype. Subtle and infrequent nucleotide changes also have been associated with cancer and other genetic diseases.

Conventional nucleotide sequencing is accomplished through bulk techniques. For example, the two most common techniques for sequencing are the Maxam and Gilbert selective chemical degradation technique and the Sanger dideoxy sequencing technique. Bulk sequencing techniques are not useful for the identification of subtle or rare nucleotide changes due to the many cloning, amplification and electrophoresis steps that complicate the process of gaining useful information regarding individual nucleotides. As such, research has evolved toward methods for rapid sequencing, such as single molecule sequencing technologies. The ability to sequence and gain information from single molecules obtained from an individual patient is the next milestone for genomic sequencing. However, effective diagnosis and management of important diseases through single molecule sequencing is impeded by lack of cost-effective tools and methods for screening individual molecules.

A number of nucleic acid polymerases have been isolated and purified from mesophilic and thermophilic organisms and applied to bulk sequencing, which utilizes amplification by polymerase chain reaction. Due to the denaturation cycle of polymerase chain reaction, a greater number of thermophilic polymerases have been investigated for their thermostable properties at high temperatures, which generally are greater than 70° C. For example, DNA polymerases have been isolated from thermophilic bacteria that are capable of growth at very high temperatures including Bacillus steraothermophilus that have a half-life of 15 minutes at 87°.

Thus, there exists a need in the art to develop nucleic acid polymerases and methods of using nucleic acid polymerases that are cost-effective and successful for single molecule nucleic acid sequencing and analysis.

### SUMMARY OF THE INVENTION

The invention provides for the use of thermophillic polymerases in single molecule sequencing reactions. It has been discovered that thermophillic polymerases, which traditionally have been used for amplification reactions at high temperature (due to their thermostability), are highly-effective at lower temperatures in single molecule sequencing reactions. In a preferred embodiment, polymerization takes place at a temperature of between about 20° C. to about 70° C.

Preferred methods of the invention comprise conducting a single molecule sequencing reaction in the presence of a thermophilic polymerase. Single molecule sequencing according to the invention comprises template-dependent nucleic acid synthesis. In a preferred embodiment, nucleic acid primers are exposed to template molecules having a primer binding site. Polymerase then directs the extension of the primer in a template-dependent fashion in the presence of labeled nucleotides or nucleotide analogs. According to the invention, primers are support-bound in a manner that allows unique optical identification of signaling events from the labeled nucleotide or nucleotide analogs as they are incorporated into the growing primer strand. In preferred methods of the invention, the thermophilic polymerase used in sequencing reactions is a 9° N DNA polymerase or a variant of the 9° N DNA polymerase. For example, a preferred variant of the 9° N DNA polymerase is an Archaeon polymerase with enhanced ability to incorporate modified nucleotides, such as a 9° N A485L (exo-) DNA polymerase. Preferred polymerases have a reduced 3′ to 5′ proofreading activity (exo-).

Methods according to the present invention comprise exposing a target nucleic acid molecule and a polymerase and at least one nucleotide or nucleotide analog to each other under non-elevated temperature or ambient temperature. With bulk sequencing of nucleic acids, thermophilic polymerases are required for their thermostable properties at elevated temperatures necessary for amplification when conducting a polymerase chain reaction. However, methods according to the invention include conducting a primer extension at lower or ambient temperatures, such as a temperature of about 20-70° C. In some embodiments, primer extension is conducted at a temperatures of about 20-50° C., about 30-40° C., or preferably about 37° C.

Methods according to the invention also comprise exposing a target nucleic acid to a primer and thermophilic polymerase to incorporate a modified nucleotide for extension of the primer. A modified nucleotide includes any nucleotide analog, such as a dideoxynucleotide, a ribonucleotide, and an acyclonucleotide. A modified nucleotide also can be a non-chain terminating nucleotide such as, for example, a deoxynucleotide including dATP, dTTP, dUTP, dCTP, and dGTP. In general, however, a modified nucleotide includes any base or modified base that exhibits Watson-Crick base pairing. Examples of nucleotide analogs include any modified base or synthetic analog such as, for example, a 7-deaxa-adenine, a 7-deaxa-guanine, inosine, xanthine, AMP, GMP, guanosine.

In some embodiments, a nucleotide analog comprises a removable linker. Also, a nucleotide analog can be modified to remove, cap, or modify the 3′ hydroxyl group. By so doing the 3′ hydroxyl group from the incorporated nucleotide in the primer, further extension is halted or impeded. In certain embodiments, the modified nucleotide is engineered so that the 3′ hydroxyl group can be removed and/or added by chemical methods.

Preferred methods of the invention comprise optically detecting incorporation of a nucleotide or nucleotide analog in a template-dependent primer extension reaction. In preferred embodiments, nucleotides are labeled for detection, preferably with a fluorescent label. In one embodiment, methods of the invention comprise detecting coincident fluorescence emission of a first fluorescent label and a second fluorescent label. The labels are attached to the polymerase and to the nucleotide base to be added. Coincident fluorescence emission preferably occurs between about 400 nm and about 900 nm.

There are many detectable labels appropriate for use with the methods of the invention. Any optically-detectable label is useful in methods of the invention. Especially preferred are fluorescent labels and dyes. For example, rhodamine, BODIPY, alexa, or any other conjugated dye is used in order to facilitate optical detection of individual nucleotides. In certain preferred embodiments, a detectable label is selected from cyanine 5 and cyanine 3.

Methods according to the invention also comprise removing or neutralizing a label subsequent to detecting it. Generally, a plurality of target nucleic acids is attached to a substrate or an array. Each member of the plurality is attached to a surface, such as glass or fused silica, preferably by covalent attachment. One skilled in the art understands that target nucleic acids can be attached to any surface that allows for primer extension, and preferably, to any surface suitable for detecting incorporation of nucleotides or nucleotide analogs. As such, in some embodiments, each member of the plurality of target nucleic acids is covalently attached to a surface that has reduced background fluorescence with respect to glass, polished glass or fused silica. Examples of surfaces appropriate for the invention include polytetrafluoroethylene or a derivative of polytetrafluoroethylene, such as silanized polytetrafluoroethylene. In addition, in preferred embodiments of the invention target nucleic acids are spaced apart on a substrate such that each target is optically resolvable. In practice, for example, the target may be optically resolved by detecting a fluorescent label attached to the nucleotide.

When conducting a primer extension reaction, after detecting the incorporation of a label, preferred methods according to the invention comprise the step of washing unincorporated reagents, such as nucleotides, nucleotide analogs, labels, dyes and/or buffer from the substrate. In certain embodiments, methods according to the invention provide-for neutralizing a label by photobleaching. This may be accomplished by focusing a laser with a short laser pulse, for example, for a short duration of time with increasing laser intensity. In other embodiments, a label may be photocleaved. For example, a light-sensitive label bound to a nucleotide may be photocleaved by focusing a particular wavelength of light on the label. Generally, it may be preferable to use lasers having differing wavelengths for exciting and photocleaving. Labels may be removed from a substrate using reagents, such as NaOH or other appropriate buffer reagent.

A detailed description of embodiments of the invention is provided below. Other embodiments of the invention are apparent upon review of the detailed description that follows.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts the comparison of the activity of the 9° N A485L (exo-) DNA polymerase and the Vent (exo-) polymerase using Cy3-dNTPs as a function of relative fluorescence units over a period of time.

FIG. 2 depicts the comparison of the activity of the 9° N A485L (exo-) DNA polymerase and the Vent (exo-) polymerase using Cy5-dNTPs as a function of relative fluorescence units over a period of time.

### DETAILED DESCRIPTION OF THE INVENTION

Single molecule sequencing requires highly-sensitive and cost-effective tools to provide rapid and accurate results. Single molecule sequencing has the potential to provide sequence-specific genomic information that is relevant to both normal and diseased function. Among the tools on which sequencing reactions are most dependent are polymerase enzymes.

The present invention provides for use in low temperature single molecule sequencing thermophilic polymerases that were developed for bulk sequencing reactions that cycle through high amplification temperatures. One example of a thermophilic polymerase that traditionally is used for its thermostable properties at elevated temperatures is the 9 degrees north A485L (exo-) DNA polymerase. According to the invention, these thermophilic polymerases are useful in single molecule reactions conducted at lower temperatures that are typically thought to be optimal for the enzyme. The polymerase 9° N (exo-) /A485L is sold commercially by New England BioLabs (Beverly, Mass.) as Therminator™ and by Perkin-Elmer (Boston, Mass.) in AcycloPrime SNP kits as AcycloPol™. Generally, the variant of the 9° N DNA polymerase is isolated and purified from an E. coli strain that carries the 9° N A485L (exo-) DNA Polymerase gene, a genetically engineered form of the native DNA polymerase from Thermococcus species 9° N-7. In addition, the 9° N DNA polymerase and/or variant thereof can be purified free of contaminating endonucleases and exonucleases.

Generally, amplification and cloning steps that are involved in polymerase chain reaction require providing thousands of copies of nucleic acids under denaturation conditions that expose the polymerase to high temperatures, such as temperatures greater than about 70° C. To meet the need for thermostability at elevated temperatures required in traditional polymerase chain reaction techniques, technicians and researchers have identified thermophilic polymerases that are thermostable and, therefore, retain their ability to incorporate nucleotides in a primer in elevated temperature conditions. However, methods according to the present invention utilize these thermophilic polymerases in primer extension reactions at non-elevated temperatures for sequencing single molecules. Accordingly, methods of the invention include conducting a primer extension reaction at a temperature of about 20-70° C. In some embodiments, primer extension using thermophilic polymerases, such as the 9 degrees north A485L (exo-) DNA polymerase, is conducted at a temperatures of about 20-50° C., at about 30-40° C., or preferably at about 37° C.

Without the wasteful and expensive cloning and amplification steps required in current DNA sequencing technologies, methods according to the invention provide for simpler and less error-prone sequencing with greater applications in disease detection and diagnosis for individual analysis. Such methods are particularly useful in connection with a variety of biological samples, such as blood, urine, cerebrospinal fluid, seminal fluid, saliva, breast nipple aspirate, sputum, stool and biopsy tissue. Especially preferred are samples of luminal fluid because such samples are generally free of intact, healthy cells. However, any tissue or body fluid specimen may be used according to methods of the invention.

Nevertheless, the target nucleic acid can come from a variety of sources. For example, nucleic acids can be naturally occurring DNA or RNA isolated from any source, recombinant molecules, cDNA, or synthetic analogs, as known in the art. For example, the target nucleic acid may be genomic DNA, genes, gene fragments, exons, introns, regulatory elements (such as promoters, enhancers, initiation and termination regions, expression regulatory factors, expression controls, and other control regions), DNA comprising one or more single-nucleotide polymorphisms (SNPs), allelic variants, and other mutations. Also included is the full genome of one or more cells, for example cells from different stages of diseases such as cancer. The target nucleic acid may also be mRNA, tRNA, rRNA, ribozymes, splice variants, antisense RNA, and RNAi. Also contemplated according to the invention are RNA with a recognition site for binding a polymerase, transcripts of a single cell, organelle or microorganism, and all or portions of RNA complements of one or more cells, for example, cells from different stages of development or differentiation, and cells from different species. Nucleic acids can be obtained from any cell of a person, animal, plant, bacteria, or virus, including pathogenic microbes or other cellular organisms. Individual nucleic acids can be isolated for analysis.

Methods according to the invention provide for the determination of the sequence of a single molecule, such as a target nucleic acid. Generally, target nucleic acids can have a length of about 5 bases, about 10 bases, about 20 bases, about 30 bases, about 40 bases, about 50 bases, about 60 bases, about 70 bases, about 80 bases, about 90 bases, about 100 bases, about 200 bases, about 500 bases, about 1 kb, about 3 kb, about 10 kb, or about 20 kb and so on. Methods according to the invention include exposing a target nucleic acid to a primer. In general, the primer is complementary to at least a portion of the target nucleic acid. The target nucleic acid also is exposed to a thermophilic polymerase (as discussed herein) and at least one nucleotide or nucleotide analog allowing for extension of the primer. A nucleotide or nucleotide analog includes any base or base-type including adenine, cytosine, guanine, uracil, or thymine bases. In addition, additional nucleotide analogs include xanthine or hypoxanthine, 5-bromouracil, 2-aminopurine, deoxyinosine, or methylated cytosine, such as 5-methylcytosine, N4-methoxydeoxycytosine, and the like. Also included are bases of polynucleotide mimetics, such as methylated nucleic acids, e.g., 2′-O-methRNA, peptide nucleic acids, modified peptide nucleic acids, and any other structural moiety that can act substantially like a nucleotide or base, for example, by exhibiting base-complementarity with one or more bases that occur in DNA or RNA and/or being capable of base-complementary incorporation.

Methods of the invention also include detecting incorporation of the nucleotide or nucleotide analog in the primer and, repeating the exposing, conducting and/or detecting steps to determine a sequence of the target nucleic acid. By using the right tools in single molecule sequencing, a researcher can compile the sequence of a complement of the target nucleic acid based upon sequential incorporation of the nucleotides into the primer. Similarly, the researcher can compile the sequence of the target nucleic acid based upon the complement sequence.

Also, a nucleotide analog can be modified to remove, cap or modify the 3′ hydroxyl group. As such, in certain embodiments, methods of the invention can include, for example, the step of removing the 3′ hydroxyl group from the incorporate nucleotide or nucleotide analog. By removing the 3′ hydroxyl group from the incorporated nucleotide in the primer, further extension is halted or impeded. In certain embodiments, the modified nucleotide can be engineered so that the 3′ hydroxyl group can be removed and/or added by chemical methods.

In addition, a nucleotide analog can be modified to include a moiety that is sufficiently large to prevent or sterically hinder further chain elongation by interfering with the polymerase, thereby halting incorporation of additional nucleotides or nucleotide analogs. Subsequent removal of the moiety, or at least the steric-hindering portion of the moiety, can concomitantly reverse chain termination and allow chain elongation to proceed. In some embodiments, the moiety also can be a label. As such, in those embodiments, chemically cleaving or photocleaving the blocking moiety may also chemically-bleach or photo-bleach the label, respectively.

The nucleic acids suitable for analysis with the invention can be DNA or RNA, as discussed herein. The methods according to the invention can provide de novo sequencing, sequence analysis, DNA fingerprinting, polymorphism identification, for example single nucleotide polymorphisms (SNP) detection, as well as applications for genetic cancer research. Applied to RNA sequences, methods according to the invention also can identify alternate splice sites, enumerate copy number, measure gene expression, identify unknown RNA molecules present in cells at low copy number, annotate genomes by determining which sequences are actually transcribed, determine phylogenic relationships, elucidate differentiation of cells, and facilitate tissue engineering. The methods according to the invention also can be used to analyze activities of other biomacromolecules such as RNA translation and protein assembly. Certain aspects of the invention lead to more sensitive detection of incorporated signals and faster sequencing.

Methods of the invention also include conducting primer extension reactions with target nucleic acids that are attached to a substrate, surface, support or an array. Each member of the plurality of target nucleic acids can be covalently attached to a surface including glass or fused silica. For example, each member of the plurality of target nucleic acids can be covalently attached to a surface that has reduced background fluorescence with respect to glass, polished glass, fused silica or plastic. Examples of surfaces appropriate for the invention include, for example, polytetrafluoroethylene or a derivative of polytetrafluoroethylene, such as silanized polytetrafluoroethylene.

Locations on a substrate, surface, support or array include a target nucleic acid that is linked thereto. In some embodiments, the locations include a primer, a target polynucleotide-primer complex, and/or a polymerase bound thereto. These moieties can be bound or immobilized on the surface of the substrate or array by covalent bonding, non-covalent bonding, ionic bonding, hydrogen bonding, van der Waals forces, hydrophobic bonding, or a combination thereof. The immobilizing may utilize one or more binding-pairs, including, but not limited to, an antigen-antibody binding pair, a streptavidin-biotin binding pair, photoactivated coupling molecules, and a pair of complementary nucleic acids. Furthermore, the substrate or support may include a semi-solid support (e.g., a gel or other matrix), and/or a porous support (e.g., a nylon membrane or other membrane). The surface of the substrate or support may be planar, curved, pointed, or any suitable two-dimensional or three-dimensional geometry.

A single molecule substrate or array describes a support or an array in which all or a subset of molecules of the array can be individually resolved and/or detected. According to invention, methods include the step of detecting incorporation of a nucleotide or nucleotide analog in a primer. Generally, the detection system includes any device that can detect and/or record light emitted from a nucleotide, from a target nucleic acid and/or a primer, and/or a polymerase. Accordingly, a detection system has single-molecule resolution or the ability to resolve one molecule from another. For example, in certain embodiments, the detection limit is in the order of a micron. Therefore, two molecules can be a few microns apart and be resolved, that is individually detected and/or detectably distinguished from each other.

Certain embodiments of the invention are described in the following examples, which are not meant to be limiting.

### EXAMPLES

Experiments were conducted to determine whether thermophilic polymerases are capable of incorporating nucleotides in primer extension reactions for single molecule sequencing. Various thermophilic polymerases were screened, including the 9 degrees north A485L (exo-) DNA polymerase, and exposed to fluorescently labeled nucleotides.

### Example 1

Incorporation of Nucleotides using Polymerases in Single Molecule Sequencing

A target nucleic acid is obtained from a patient using a variety of known procedures for extracting the nucleic acid. Although unnecessary for single molecule sequencing, the extracted nucleic acid can be optionally amplified to a concentration convenient for genotyping or sequence work. Nucleic acid amplification methods are known in the art, such as polymerase chain reaction. Other amplification methods known in the art that can be used include ligase chain reaction, for example.

The single stranded plasmid can be primed by 5′-biotinylated primers, and double stranded plasmid can then be synthesized. The double stranded plasmid can then be linearized, and the biotinylated strand purified. Analyzing a target nucleic acid by synthesizing its complementary strand may involve hybridizing a primer to the target nucleic acid. The primer can be selected to be sufficiently long to prime the synthesis of extension products in the presence of a thermophilic polymerase, such as a variant of 9° N DNA polymerase (9 degrees north A485L (exo-) DNA polymerase). Primer length can be selected to facilitate hybridization to a sufficiently complementary region of the template polynucleotide downstream of the region to be analyzed. The exact lengths of the primers depend on many factors, including temperature, source of primer.

If part of the region downstream of the sequence to be analyzed is known, a specific primer can be constructed and hybridized to this region of the target nucleic acid. Alternatively, if sequences of the downstream region on the target nucleic acid are not known, universal or random primers may be used in random primer combinations. As another approach, a linker or adaptor can be joined to the ends of a target nucleic acid polynucleotide by a ligase and primers can be designed to bind to these adaptors. That is, a linker or adaptor can be ligated to at least one target nucleic acid of unknown sequence to allow for primer hybridization. Alternatively, known sequences may be biotinylated and ligated to the targets. In yet another approach, nucleic acid may be digested with a restriction endonuclease, and primers designed to hybridize with the known restriction sites that define the ends of the fragments produced.

Primers can be synthetically made using conventional nucleic acid synthesis techniques. For example, primers can be synthesized on an automated DNA synthesizer, e.g. an Applied Biosystems, Inc. (Foster City, Calif.) model 392 or 394 DNA/RNA Synthesizer, using standard chemistries, such as phosphoramidite chemistry, and the like. Alternative chemistries, e.g., resulting in non-natural backbone groups, such as phosphorothioate, phosphoramidate, and the like, may also be employed provided that, for example, the resulting oligonucleotides are compatible with the polymerizing agent. The primers can also be ordered commercially from a variety of companies which specialize in custom nucleic acids such as Operon Inc (Alameda, Calif.).

In some instances, the primer can include a label. When hybridized to a linked nucleic acid molecule, the label facilitates locating the bound molecule through imaging. The primer can be labeled with a fluorescent labeling moiety (e.g., Cy3 or Cy5), or any other means used to label nucleotides. The detectable label used to label the primer can be different from the label used on the nucleotides or nucleotide analogs used on the nucleotides in the subsequent extension reactions. Suitable fluorescent labels include, but are not limited to, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodarnine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine.

If the target polynucleotide-primer complex is to be linked on a surface of a substrate or array, the primer can be hybridized before or after such linking. Primer annealing can be performed under conditions which are stringent enough to require sufficient sequence specificity, yet permissive enough to allow formation of stable hybrids at an acceptable rate. The temperature and time required for primer annealing depend upon several factors including base composition, length, and concentration of the primer; the nature of the solvent used, e.g., the concentration of DMSO, formamide, or glycerol; as well as the concentrations of counter ions, such as magnesium. Typically, hybridization with synthetic polynucleotides is carried out at a temperature that is approximately 5° C. to approximately 10° C. below the melting temperature (Tm) of the target polynucleotide-primer complex in the annealing solvent. However, according to methods of the invention, hybridization may be performed at much lower temperatures, such as for example 30-50° C. or 30-40° C. The annealing reaction can be complete within a few seconds.

After preparing the target nucleic acid and optionally linking it on a substrate, primer extension reactions can be performed to analyze the target polynucleotide sequence by synthesizing its complementary strand. The primer is extended by a thermophilic polymerase in the presence of a nucleotide or nucleotide analog bearing a detectable label at a temperature of about 10 to about 70° C., about 20 to about 60° C., about 30 to about 50° C., or preferably at about 37° C. In other embodiments, two, three or all four types of nucleotides are present, each bearing a detectably distinguishable label. In some embodiments of the invention, a combination of labeled and non-labeled nucleotides or nucleotide analogs are used in the primer extension reaction for analysis.

Depending on the template, a DNA polymerase, an RNA polymerase, or a reverse transcriptase can be used in the primer extension reactions. Preferably, a thermophilic polymerase is used according to the invention. And more preferably, a 9° N DNA polymerase or variant thereof is used as the polymerizing agent. For example, in one embodiment, a variant of the 9° N DNA polymerase that is an Archaeon polymerase with enhanced ability to incorporate a modified nucleotide can be used in the primer extension reaction at a temperature of about 37° C. An Archaeon polymerase may be a 9 degrees north A485L (exo-) DNA polymerase, for example. Generally, the polymerase according to the invention has high incorporation accuracy and a processivity (number of nucleotides incorporated before the polymerase dissociates from the target nucleic acid) of at least about 20 nucleotides. Nucleotides can be selected to be compatible with the polymerase, for example, the 9 degrees north A485L (exo-) DNA polymerase.

The incorporation of the labeled nucleotide or nucleotide analog can be detected on the primer. A number of systems are available to accomplish this. Methods for visualizing single molecules of labeled nucleotides with an intercalating dye include, e.g., fluorescence microscopy. In some embodiments, the fluorescent spectrum and lifetime of a single molecule excited-state can be measured. Standard detectors such as a photomultiplier tube or avalanche photodiode can be used. Full field imaging with a two-stage image intensified CCD camera can also used. Additionally, low noise cooled CCD can also be used to detect single fluorescent molecules.

The detection system for the signal may depend upon the labeling moiety used, which can be defined by the chemistry available. For optical signals, a combination of an optical fiber or charged couple device (CCD) can be used in the detection step. In the embodiments where the substrate is itself transparent to the radiation used, it is possible to have an incident light beam pass through the substrate with the detector located opposite the substrate from the primer. For electromagnetic labels, various forms of spectroscopy systems can be used. Various physical orientations for the detection system are available and known in the art.

A number of approaches can be used to detect incorporation of fluorescently-labeled nucleotides into a single molecule. Optical systems include near-field scanning microscopy, far-field confocal microscopy, wide-field epi-illumination, light scattering, dark field microscopy, photoconversion, single and/or multiphoton excitation, spectral wavelength discrimination, fluorophore identification, evanescent wave illumination, and total internal reflection fluorescence (TIRF) microscopy. In general, methods involve detection of laser-activated fluorescence using a microscope equipped with a camera, sometimes referred to as high-efficiency photon detection system. Suitable photon detection systems include, but are not limited to, photodiodes and intensified CCD cameras. For example, an intensified charge couple device (ICCD) camera can be used. The use of an ICCD camera to image individual fluorescent dye molecules in a fluid near a surface provides numerous advantages. For example, with an ICCD optical setup, it is possible to acquire a sequence of images (movies) of fluorophores.

### Example 2

Determining Processivity of 9° N A485 (exo-) DNA Polymerase in the Presence of Labeled Nucleotides

As a proof-of-principle to determine whether the 9° N A485 (exo-) DNA polymerase accurately incorporates labeled nucleotides into the primer, an extension experiment can be performed in a test tube rather than on a substrate. In this experiment, incorporation of dCTP-Cy3 and a polymerization terminator, ddCTP, can be detected using a 7G DNA template (a DNA strand having a G residue every 7 bases). The annealed primer is extended in the presence of non-labeled dATP, dGTP, dTTP, Cy3-labeled dCTP, and ddCTP. The ratio of Cy3-dCTP and ddCTP can be analyzed. The reaction products can be separated on a gel, fluorescence can be excited, and the signals detected, using an automatic sequencer, such as, ABI-377.

The presence of fluorescence intensity from primer extension products of various lengths which were terminated by incorporation of ddCTP at the different G residues in the 7G oligomer template can be analyzed, for example, on a gel. Bands correlating to extension products suggest the incorporation of nucleotides, and the different bands suggest incorporation of nucleotides of differing lengths.

### Example 3

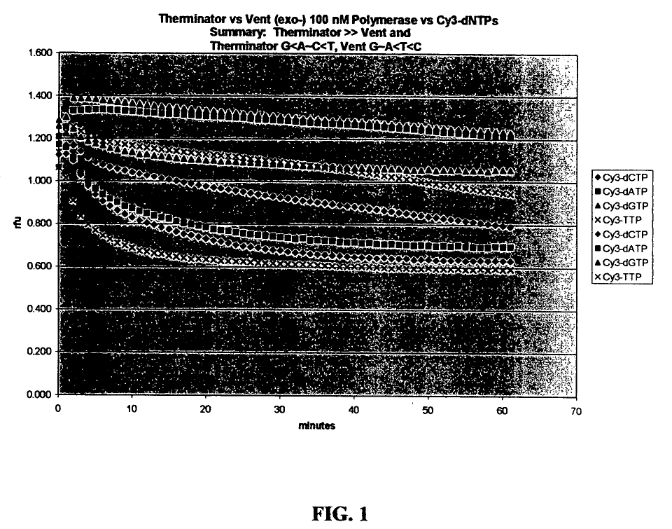

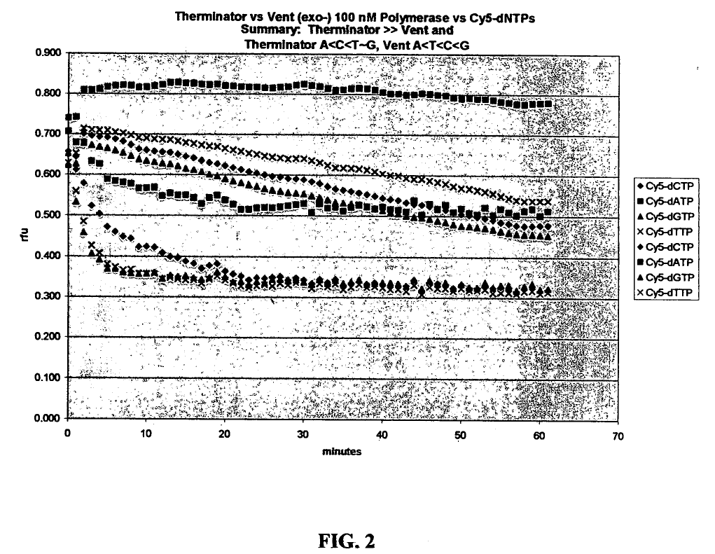

A screening process was established and the 9 degrees north A485L (exo-) DNA polymerase was tested in a bulk assay. As depicted in FIGS. 1 and 2, this polymerase was found to substantially outperform the Vent (exo-) polymerase. The 9 degrees north A485L (exo-) DNA polymerase is sold commercially by New England BioLabs (Beverly, Mass.) as “Therminator™ and by Perkin-Elmer (Boston, Mass.) in AcycloPrime SNP kits as AcycloPol™. As depicted in FIGS. 1 and 2, based upon the screening protocols, the Vent (exo-) polymerase and the 9° N A485L (exo-) DNA polymerase, which typically have optimal temperature ranges of 65-80° C. were found to perform satisfactorily at about 37° C. The activity of the 9° N A485L (exo-) DNA polymerase is shown as a function of relative fluorescence units over a period of time.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

1. A method for nucleic acid sequence determination, the method comprising the steps of:
(a) exposing a target nucleic acid to a primer that is complementary to at least a portion of the target, a thermophilic polymerase, and at least one nucleotide for extension of said primer;
(b) conducting a primer extension at a temperature of about 20-70° C.;
(c) detecting incorporation of said nucleotide in said primer; and,
(d) repeating steps (a), (b) and (c), thereby to determine a sequence of said target.

2. The method of claim 1, wherein said polymerase is a 9° N DNA polymerase.

3. The method of claim 1, wherein said polymerase is a variant of said 9° N DNA polymerase.

4. The method of claim 3, wherein said polymerase is a 9° N A485L (exo-) DNA polymerase.

5. The method of claim 1, wherein said variant is a thermostable polymerase with enhanced ability to incorporate a modified nucleotide.

6. The method of claim 5, wherein said variant is an Archaeon polymerase.

7. The method of claim 1, wherein the primer extension is conducted at a temperature of about 20-70° C.

8. The method of claim 1, wherein the primer extension is conducted at a temperature of about 30-40° C.

9. The method of claim 1, wherein the primer extension is conducted at a temperature of about 37° C.

10. The method of claim 5, wherein said modified nucleotide is a nucleotide analog.

11. The method of claim 5, wherein said nucleotide analog is selected from the group consisting of a deoxynucleotide, a ribonucleotide, and analog thereof.

12. The method of claim 5, wherein said nucleotide analog comprises a cleavable linker.

13. The method of claim 12, wherein the cleavage of said linker is done using photolysis or chemical hydrolysis.

14. The method of claim 5, wherein said nucleotide analog lacks a 3′ hydroxyl group.

15. The method of claim 14, wherein the nucleotide analog is a 2′,3′-dideoxynucleotide, acyclonucleotide, or analog thereof.

16. The method of claim 1, wherein said polymerase has a decreased 3′ to 5′ proofreading exonuclease activity.

17. The method of claim 1, wherein said nucleotide comprises a detectable label.

18. The method of claim 17, wherein said label is a fluorescent label.

19. The method of claim 18, wherein the detectable label is selected from the group consisting of cyanine, rhodamine, fluorescein, coumarin, BODIPY, alexa, or conjugated multi-dyes.

20. The method of claim 12, further comprising the step of removing or neutralizing said label subsequent to said detecting step.

21. The method of claim 1, wherein said detecting step comprises optically detecting incorporation of said nucleotide.

22. The method of claim 1, wherein said target is attached to a substrate.

23. The method of claim 1, further comprising the step of washing an unincorporated nucleotide.

24. The method of claim 22, wherein a plurality of said target nucleic acids are spaced apart such that each target is optically resolvable.

25. The method of claim 21, wherein said detecting step comprises detecting a fluorescent label attached to said nucleotide.

26. The method of claim 25, wherein said label represents a single nucleic acid molecule.

27. The method of claim 1, further comprising the step of compiling a sequence of a complement of said target based upon sequential incorporation of said nucleotides into said primer.

28. The method of claim 27, further comprising the step of compiling a sequence of said target based upon said complement sequence.

29. The method of claim 24, wherein each member of said plurality is covalently attached to a surface comprising glass or fused silica.

30. The method of claim 29, wherein each member of said plurality is covalently attached to a surface that has reduced background fluorescence with respect to polished glass or fused silica.

31. The method of claim 30, wherein said surface is polytetrafluoroethylene or a derivative of polytetrafluoroethylene.

32. The method of claim 31, wherein said derivative is silanized.

33. The method of claim 19, wherein said label is selected from a cyanine 5 dye and a cyanine 3 dye.

34. The method of claim 17, wherein said nucleotide comprises a first fluorescent label and said polymerase comprises a second fluorescent label.

35. The method of claim 34, wherein said detecting step comprises detecting coincident fluorescence emission of said first fluorescent label and said second fluorescent label.

36. The method of claim 35, wherein the coincident fluorescence emission spectrum is between about 400 nm to about 900 nm.

37. The method of claim 36, wherein said coincident detection represents the presence of a single labeled molecule.

38. The method of claim 5, wherein said nucleotide is a non-chain terminating nucleotide.

39. The method of claim 38, wherein said non-chain terminating nucleotide is a deoxynucleotide selected from the group consisting of dATP, dTTP, dUTP, dCTP, and dGTP.

40. The method of claim 38, wherein said non-chain terminating nucleotide is a ribonucleotide selected from the group consisting of ATP, UTP, CTP, and GTP.
